(19) United States
(12) Patent Application Publication
(10) Pub. No.: US 2017/0000405 A1
(43) Pub. Date: Jan. 5, 2017

(54) RAPID, REPRODUCIBLE, NON-INVASIVE PREDICTOR OF CADAVERIC DONOR LIVER GRAFT UTILIZATION

(71) Applicant: THE REGENTS OF THE UNIVERSITY OF CALIFORNIA, Oakland, CA (US)

(72) Inventor: Ali Zarrinpar, Los Angeles, CA (US)

(73) Assignee: THE REGENTS OF THE UNIVERSITY OF CALIFORNIA, Oakland, CA (US)

(21) Appl. No.: 15/204,254

(22) Filed: Jul. 7, 2016

Related U.S. Application Data
(63) Continuation of application No. PCT/US2015/012582, filed on Jan. 23, 2015.
(60) Provisional application No. 61/930,860, filed on Jan. 23, 2014.

(51) Int. Cl. A61B 5/00; A61B 5/1455; A61B 16/00

(52) U.S. Cl. CPC A61B 5/4244; A61B 16/00; A61B 5/1455; A61B 5/6826; A61B 2017/00969

(57) ABSTRACT

A method for rapid, non-invasive assessment of liver function in a brain-dead donor, prior to procurement is presented. Quantitative assessment of liver function can be measured in brain-dead donors in the local donor service area prior to organ procurement by measuring indocyanine green plasma disappearance rates (ICG-PDR). This method can be performed with devices that use a non-invasive finger probe to obtain the ICG-PDR using pulse-densitometry.

### CROSS-REFERENCE TO RELATED APPLICATIONS

This application is a 35 U.S.C. §111(a) continuation of PCT international application number PCT/US2015/012582 filed on Jan. 23, 2015, incorporated herein by reference in its entirety, which claims priority to, and the benefit of, U.S. provisional patent application Ser. No. 61/930,860 filed on Jan. 23, 2014, incorporated herein by reference in its entirety. Priority is claimed to each of the foregoing applications.

The above-referenced PCT international application was published as PCT International Publication No. WO 2015/112795 on Jul. 30, 2015, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

Not Applicable

### BACKGROUND

1. Technical Field

The technology of this disclosure pertains generally to the assessment of liver tissue function, and more particularly to the rapid quantification of liver tissue function in brain dead donors to determine if liver tissue is acceptable for transplantation.

2. Background Discussion

Liver transplantation is the standard treatment for end-stage liver disease. Although the number of listed patients continues to grow, organ availability has plateaued resulting in increasing wait-list mortality. The donor pool has been somewhat expanded through the use of living donors, cadaveric split livers, and “extended criteria donors” (ECD). Used judiciously, the liver grafts from these sources provide an opportunity for addressing the organ shortage. However, these methods also predispose recipients to poor initial graft function and/or increased long-term risk. Factors that have been shown to affect graft utilization and function include advanced donor age, hypernatremia, prolonged warm ischemia time, pressor requirements, and donation after cardiac death.

Optimizing the use of these grafts while minimizing recipient risk requires accurate and reproducible assessments of graft quality. Clinical and laboratory criteria to measure liver quality are not reliable predictors. Therefore, the gold standard for assessing livers for transplantation remains the subjective decisions of surgeons. Attempts to evaluate all possible donors leads to inefficient use of resources, either by having surgeons evaluate many livers, only some of which are suitable for transplantation, or by having surgeons evaluate too few livers, thus forgoing useable grafts.

### BRIEF SUMMARY

Using a portable, non-invasive device, indocyanine green plasma disappearance rates (ICG-PDR) can be measured in brain-dead donors in the local donor service area prior to organ procurement. This quantitative assessment of the donor's liver function can allow for an important initial assessment of a donor's liver prior to the surgeon's assessment for transplantation.

This quantitative assessment of liver function can save time and costs associated with the transportation of non-acceptable organs for transplantation. This initial evaluation may also save the surgeon time in trying to assess livers that are not functioning at optimal levels according to the ICG-PDR. Furthermore, because ICG-PDR is such a rapid test, unlike lengthy laboratory tests, it can be applied to measuring liver function repeatedly (˜20 measurements may be performed in a 24 hour period), if there is a suspicion that the liver has suffered damage before it is procured.

Non-invasive probes, such as finger probes, may be used to perform real time measurements without added risk to the donor's liver from invasive techniques. As an example, the LiMON by PULSION may be used.

In one embodiment, a threshold ICG-PDR value can be set at 18%, whereby livers with a measured ICG-PDR of at least 18% are procured for transplantation.

In another embodiment, a threshold ICG-PDR value can be set at 24%, whereby livers with a measured ICG-PDR of at least 24% are procured for transplantation.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 is a schematic diagram of how Indocyanine Green Plasma Disappearance Rates (ICG-PDR) may be measured, according to an embodiment of the described technology.

FIG. 2A is a close up view of a schematic diagram of ICG-PDR being measured using a non-invasive finger probe, according to an embodiment of the described technology.

FIG. 2B is a graph of fractional pulsatile changes in optical absorption at 805 nm.

FIG. 2C is a graph of fractional pulsatile changes in optical absorption at 905 nm.

FIG. 3 is an example graph of clearance of ICG (C_{ICG}) over time, used in the calculation of ICG-PDR.

FIG. 4A is a schematic diagram of a screen shot for a PULSION-LiMON ICG trace of an acceptable graft, according to an embodiment of the described technology.

FIG. 4B is a schematic diagram of a screen shot for a PULSION-LiMON ICG trace of a non-acceptable graft, according to an embodiment of the described technology.

FIG. 5 is a graph illustrating ICG-PDR in donors with acceptable and non-acceptable grafts.

FIG. 6 is a flow chart of the method of measuring the ICG-PDR in a brain-dead donor, according to an embodiment of the described technology.

### DETAILED DESCRIPTION

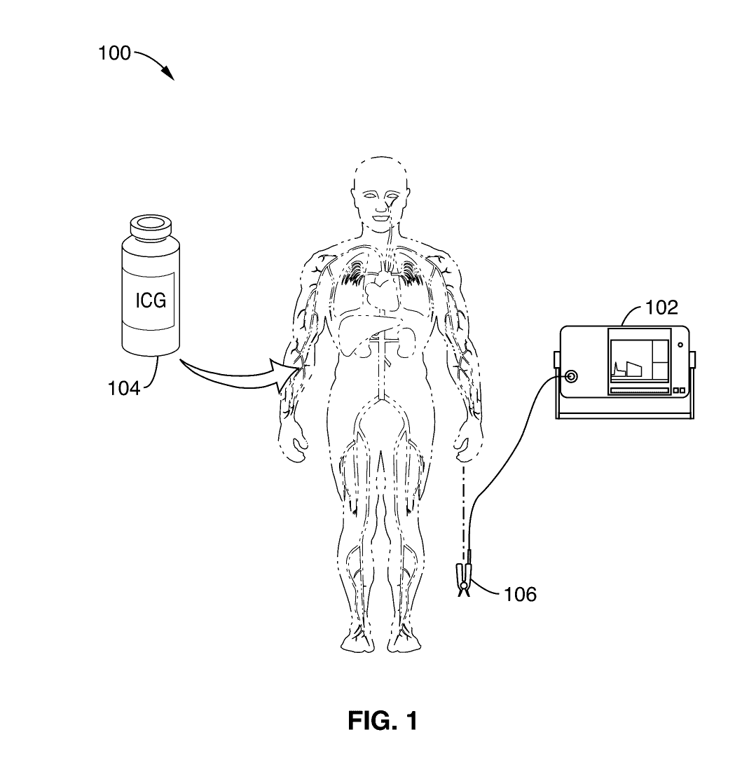

Referring more specifically to the drawings, for illustrative purposes, embodiments of the methods for assessing livers in brain-dead donors using indocyanine green plasma disappearance rates (ICG-PDR) to determine liver function before a liver is procured for graft transplantation are described herein and depicted generally in FIG. 1 through FIG. 6. It will be appreciated that the methods may vary as to the specific steps and sequence without departing from the basic concepts as disclosed herein. The method steps are merely exemplary of the order that these steps may occur. The steps may occur in any order that is desired, such that it still performs the goals of the claimed technology.

Using portable, finger-probe based devices or other similarly non-invasive devices, ICG-PDR can be measured in adult brain-dead donors in the local donor service area prior to organ procurement. The method described herein provides a quantitative technique for assessing liver function in association with liver graft utilization.

### Example

To evaluate the method for rapid, non-invasive, quantitative assessment of liver function, performed before liver graft procurement, a LiMON liver function monitor, manufactured by Pulsion Medical Systems, Munich, Germany, was used for the assessment of brain-dead donor livers by measuring Indocyanine Green Plasma Disappearance Rates (ICG-PDRs). Referring to FIG. 1, a schematic diagram 100 of how the ICG-PDR was measured in this example is presented. The LiMON monitor 102 measures Indocyanine Green (ICG) 104 clearance from the blood, after the ICG is administered intravenously to the donor, by non-invasive pulse-densitometry using, for example, a finger probe 106.

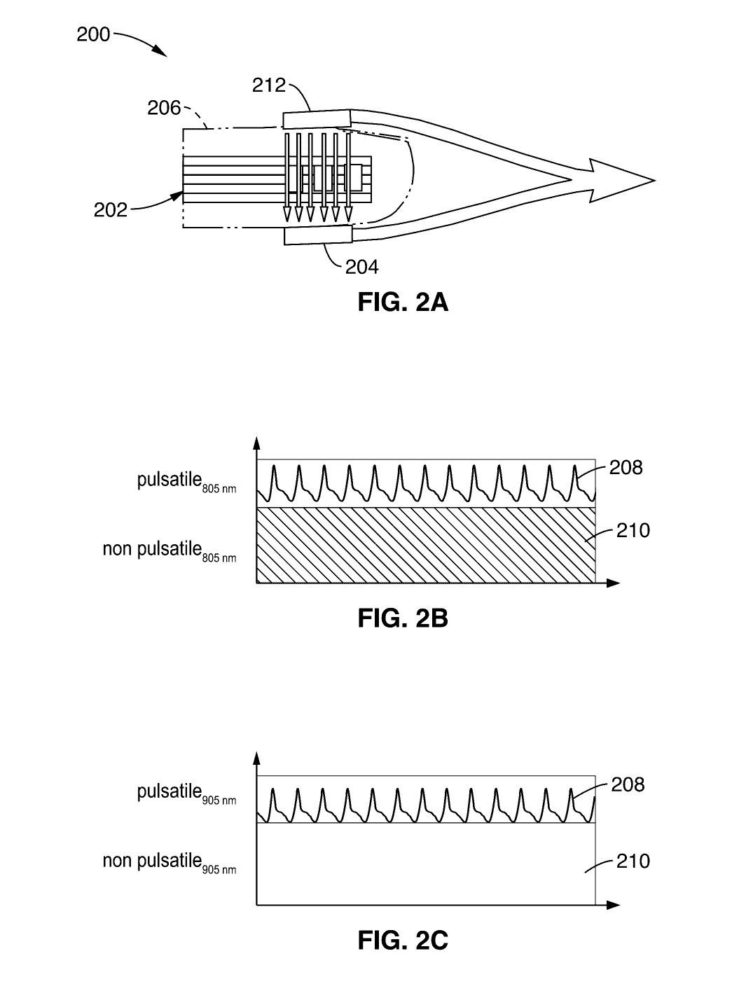

Referring to FIG. 2A through FIG. 2C, a close-up view 200 of ICG-PDR measurement by a non-invasive pulse-densitometry finger probe is presented. Injected ICG 202 is detected by a sensor 204 on the finger 206 from fractional pulsatile changes 208, 210 in optical absorption using LED lights 212. The optical peak absorption of 805 nm as shown in FIG. 2B and 905 nm as shown in FIG. 2C allows continuous measurements of ICG-PDR. The clearance of the injected dye from the blood (mg/l) can be calculated as:

$${C}_{ICG} = \frac{{pulsatile}_{905 n m} / non {pulsatile}_{905 n m}}{{pulsatile}_{805 n m} / non {pulsatile}_{805 n m}}$$

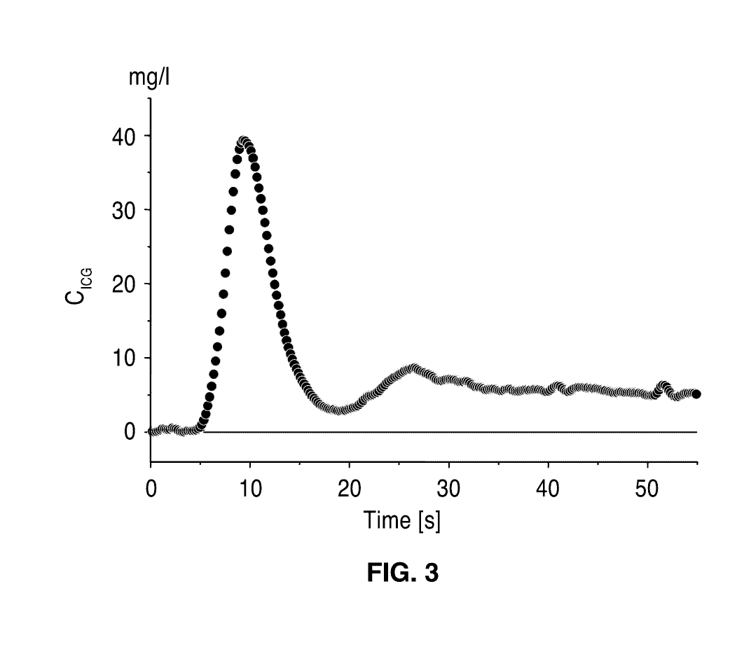

The ICG-PDR is calculated as the ICG clearance (C_{ICG}) over time, as shown in the graph in FIG. 3.

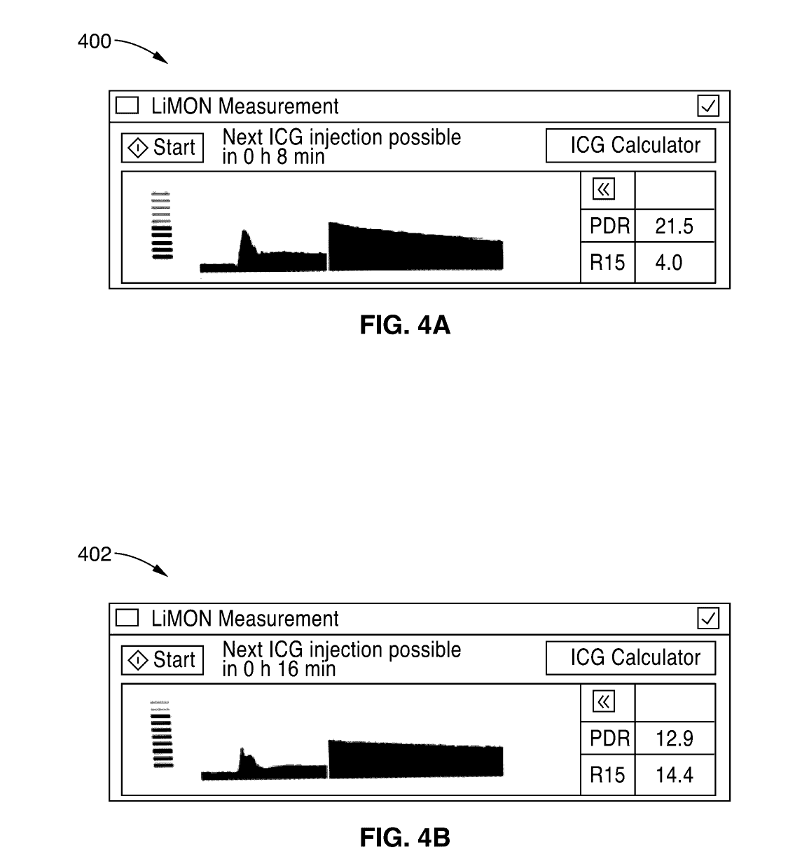

Consecutive adult brain-dead donors in the local donation service area, whose livers were offered to the study center, were assessed for liver function using the methods of the current disclosure, after permission was acquired. Immediately prior to organ procurement, indocyanine green elimination tests were conducted using the non-invasive liver function monitoring system shown in FIG. 1 and FIG. 2A. Each patient received an ICG finger clip connected to a liver function monitor. A dose of 0.25 mg/kg ICG (ICG—PULSION, Pulsion Medical Systems) was given through a central vein as a bolus and immediately flushed with 10 mL of normal saline. All of the ICG-PDR measurements were acquired using the LiMON liver function monitor manufactured by Pulsion Medical Systems, Munich, Germany. FIG. 4A shows a sample PULSION-LiMON ICG trace of an accepted liver 400 (PDR=21.5% clearance per minute). FIG. 4B shows a sample PULSION-LiMON ICG trace of a non-acceptable liver 402 (PDR=12.9% clearance per minute).

ICG-PDR measurements were performed on 53 consecutive brain-dead donors. Eleven liver grafts were declined due to quality and one was declined for size. Univariate analysis showed ICG-PDR to be the only factor associated with acceptance of an organ for transplantation. Donor Risk Index, donor age, and transaminase levels at peak or procurement were not significantly associated with utilization of liver grafts.

Additionally, there were no recipients who suffered from primary non-function and ICG-PDR was not associated with any post-operative outcome measures including POD7 AST (post-operative day 7 aspartate transaminase), ALT (alanine transaminase), Bilirubin, or INR (International Normalized Ratio—a measure of coagulopathy).

Results were correlated with surgeons' assessments and the decision to implant a graft. Both donor and recipient surgeons were blinded to ICG-PDR measurements. Post-transplantation labs of the recipient were recorded and correlated with the donor ICG-PDR.

Table 1 shows univariate predictors of liver graft utilization based on the data shown in Table 2 through Table 16. The data collected for the recipients of the liver grafts is presented in Table 2 through Table 7. The data collected for the donors of the liver grafts is presented in Table 8 through Table 16.

Table 2 shows the data for ICG-PDR, r15 (residual % of ICG at 15 minutes), Accept (graft accepted for transplant by primary center), TxAge (age of recipient at time of transplant), Gender, Expired 8/23/13 (recipient expired by 8/23/13), Dx1 (primary diagnosis) and Dx2 (secondary diagnosis) for the 53 recipients of the 53 donor liver grafts assessed. Table 3 shows the data for Race, Phys MELD (physiologic model for end stage liver disease score), INR (international normalized ratio—a measure of coagulopathy), Cr (creatinine), Tbili (total bilirubin), Plts (platelet count), Alb (albumin), and PAlb (prealbumin) for the 53 recipients of the 53 donor liver grafts assessed. Table 4 shows the data for Pressors, HD (hemodialysis), Vent (ventilator dependence), Redo (recipient is a redo liver transplant candidate), #OLT (number of liver transplant—i.e. first, second, third, etc.), L/K (combined liver/kidney transplant), HT (height), WT (weight), and BMI (body mass index) for the 53 recipients of the 53 donor liver grafts. Table 5 shows the data for Takeback# (number of times recipient was taken back to OR after transplantation), ReOLT (recipient was retransplanted after this graft), 7 d surv (7 day survival), 30 d surv (30 day survival), 6 m surv (6 month survival), 1 yr surv (1 year survival), Rpfn Bx (description of the reperfusion biopsy, and LD 1 d (lactate dehydrogenase on postoperative day 1) for the 53 recipients of the 53 donor liver grafts assessed. Table 6 shows the data for AST 1 d (aspartate transaminase on postoperative day 1), ALT 1 d (alanine transaminase on postoperative day 1), Tbili 1 d (total bilirubin on postoperative day 1), INR 1 d (INR on postoperative day 1), LD 7 d (day 7), AST 7 d (day 7), ALT 7 d (day 7), and Tbili 7 d (day 7) for the 53 recipients of the 53 donor liver grafts assessed. Table 7 shows the data for INR 7 d (day 7), LD 30 d (day 30), AST 30 d (day 30), ALT 30 d (day 30), Tbili 30 d (day 30), and INR 30 d (day 30), Age, and Male for the 53 recipients of the 53 donor liver grafts assessed.

For the 53 donor livers assessed, Table 8 shows the data for the donors' Age, Male, Race, ABO (blood type), Wt (weight), and Ht (height). Table 9 shows the data for BMI, Cause of Death, LOH (length of hospitalization), and Dopa (Dopamine) for each of the 53 donors assessed. Table 10 shows the data for Vaso (vasopressin), NE (norepinephrine), Phenyl (phenylephrine), Total Pressors, Insulin, DM (diabetes mellitus) and HTN (hypertension) for each of the 53 donors assessed. Table 11 shows the data for EtOH Abuse, Other Drugs, HgbA1c (Hemoglobin A1c level), HepB Core and HepC for the 53 donors assessed. Table 12 shows the data for whether the donor suffered Cardiac Arrest, Respiratory Arrest, the Total Downtime, CPR Duration and MAP range (mean arterial pressure range). Table 13 shows the data for MAP at Procurement of the liver grafts, Na Peak (sodium at peak), Na Proc (sodium at procurement), Cr Peak, Cr Proc, (creatinine at procurement) and TBili Peak (peak total bilirubin). Table 14 shows the data for TBili Proc (total bilirubin at procurement), AST Peak, AST Proc, ALT Peak and ALT Proc for each of the 53 donors assessed. Table 15 shows the data for INR Peak, INR Procurement, pH at Admission, pH at Procurement, pCO₂ at Admission and pCO₂ at Procurement for each of the 53 donors assessed. Finally, Table 16 shows the data for pO₂ at time of Admission, pO₂ at Procurement, HCO₃ at time of Admission, HCO₃ at time of Procurement, whether pO₂ was less than 60 and whether the donor's liver was Abnormal on Imaging.

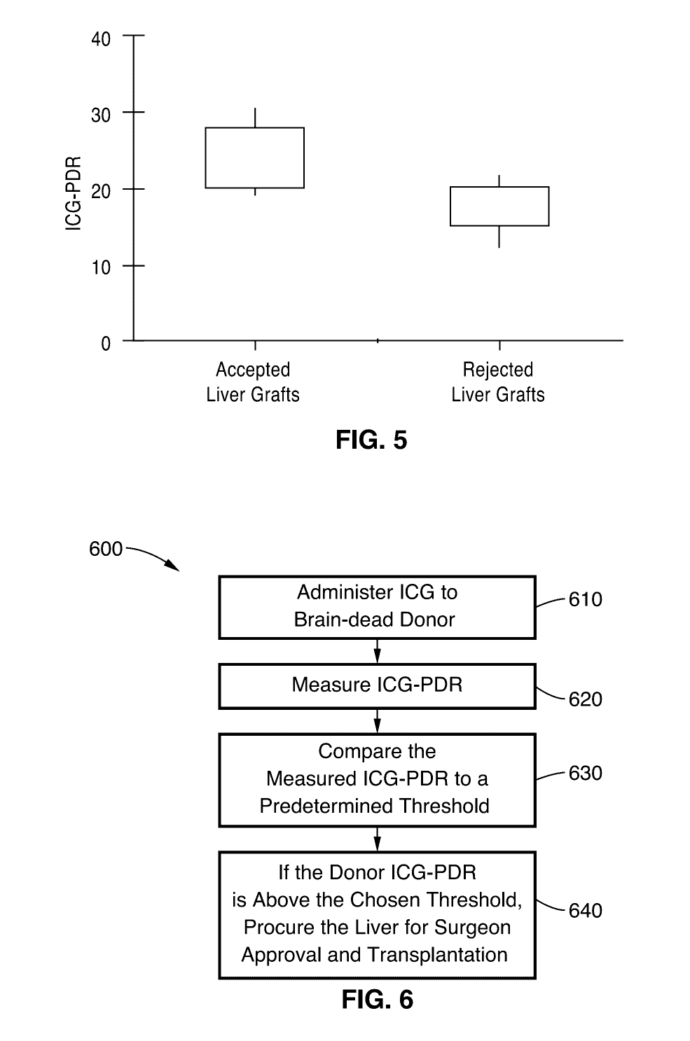

FIG. 5 is a graph that illustrates ICG-PDR in donors with accepted and rejected liver grafts according to their ICG-PDR.

Referring to FIG. 6, a flow chart 600 that details a preferred embodiment of the disclosed method is presented. At box 610, ICG is administered to the brain-dead organ donor. Indocyanine green (ICG) is a water-soluble anionic compound. It can be administered intravenously and binds mainly albumin and β-lipoproteins in the plasma. ICG is selectively absorbed by hepatocytes, independent of adenosine triphosphate (ATP), and is later excreted unaltered into the bile via an ATP-dependent transport system. It is not metabolized and does not undergo enterohepatic recirculation. Thus, ICG is particularly useful for the assessment of liver function.

At box 620, the ICG-PDR is measured using a device such as the LiMON liver function monitor. The measurement is preferably performed non-invasively, such as with the finger probe sensor previously described. Once the ICG-PDR is measured, the result is compared to a threshold value 630. If the ICG-PDR falls below the threshold, the liver can be rejected for graft transplant. The surgeon can set a particular threshold and then procure and evaluate only those donors whose ICG-PDR meets the selected threshold 640.

The successful use of a portable, quantitative means of assessing liver function in association with graft utilization is described. The data collected thus far warrant further exploration in a variety of settings to evaluate acceptable values for donated organs. At a time of increasing regional and national organ sharing, the methods described herein can assist in increasing liver graft utilization while decreasing travel risk and expense.

From the description herein, it will be appreciated that that the present disclosure encompasses multiple embodiments which include, but are not limited to, the following:

1. A method for determining whether liver tissue will be acceptable for transplantation, the method comprising: (a) measuring indocyanine green plasma disappearance rates (ICG-PDR) in a brain-dead donor prior to organ procurement; and (b) designating liver tissue in the donor as acceptable for transplantation if the ICG-PDR is greater than about 18%.

2. The method of any preceding embodiment, further comprising designating the liver tissue as acceptable for transplantation if the ICG-PCR is greater than about 24%.

3. The method of any preceding embodiment, wherein ICG-PDR is measured non-invasively.

4. The method of any preceding embodiment, wherein ICG-PDR is measured using a PULSON LiMON liver function monitor.

5. A method of determining whether liver tissue will be acceptable for transplantation, the method comprising: (a) measuring indocyanine green plasma disappearance rates (ICG-PDR) in a brain-dead donor prior to organ procurement; (b) comparing measured ICG-PDR to a threshold; and (c) designating liver tissue in the donor as acceptable for transplantation if the ICG-PDR exceeds the threshold.

6. The method of any preceding embodiment, wherein the threshold is about 18%.

7. The method of any preceding embodiment, wherein the threshold is about 24%.

8. The method of any preceding embodiment, wherein ICG-PDR is measured non-invasively.

9. The method of any preceding embodiment, wherein ICG-PDR is measured using a PULSON LiMON liver function monitor.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Univariate Predictors of Liver Graft Utilization

| | Mean (SD) | | |
|---|---|---|---|
| | Rejected | Accepted | P |
| IGG-PDR (%/min) | 17.9 (4.7) | 24.1 (6.7) | 0.006 |
| Donor pH at Admission | 7.40 (0.12) | 7.31 (0.41) | 0.06 |
| Donor Length of | 18 (36) | 6.4 (5.8) | 0.06 |
| Hospitalization (days) | | | |
| Donor Vasopressors (#) | 2 (0.77) | 1.6 (0.69) | 0.10 |
| Procurement ALT (U/L) | 65 (39) | 48 (47) | 0.28 |
| Peak ALT(U/L) | 98 (73) | 114 (186) | 0.77 |
| Donor Age (years) | 47.9 (15.3) | 43.8 (16.3) | 0.48 |
| Donor Risk Index | 1.53 (0.39) | 1.56 (0.40) | 0.82 |

TABLE 2

| | ICG- | | | | | Expired | | |
|---|---|---|---|---|---|---|---|---|
| Recipient | PDR | r15 | Accept | TxAge | Gender | Aug. 23, 2013 | Dx1 | Dx2 |
| 1 | no | no | 0 | 25 | F | 1 | Chronic | |
| | read | read | | | | | Rjxn | |
| 2 | 23.1 | 3.1 | 0 | 25 | F | 1 | Chronic | |
| | | | | | | | Rjxn | |
| 3 | no | no | 0 | 27 | M | 0 | 9930 | 9940 |
| | read | read | | | | | | |
| 4 | 18.3 | 6.4 | 0 | 56 | M | 0 | HepC | HCC |
| 5 | 12.6 | 15.2 | 0 | 68 | M | 0 | 4202 | HCC |
| 6 | 16.9 | 7.9 | 0 | 24 | F | 0 | 4212 | |
| 7 | 14.1 | 12.1 | 0 | 68 | F | 0 | NASH | HCC |
| 8 | 16.3 | 8.7 | 0 | 49 | M | 1 | EtOH | |
| 9 | 27.7 | 1.6 | 0 | 45 | M | 0 | HepB | |
| 10 | 22.2 | 3.6 | 0 | 46 | M | 0 | HepC | HCC |
| 11 | size | size | 0 | 74 | F | 0 | EtOH | |
| 12 | 16.5 | 8.4 | 0 | 51 | M | 1 | HepC | HCC |
| 13 | 12.4 | 15.6 | 0 | 62 | M | 0 | HepC | HCC |
| 14 | 17.3 | 7.5 | 0 | 66 | M | 0 | 4213 | |
| 15 | no | no | 0 | 60 | M | 0 | Chronic | |
| | read | read | | | | | Rjxn | |
| 16 | 28.7 | 1.4 | 1 | 52 | M | 0 | HepC | HCC |
| 17 | 28.7 | 1.4 | 1 | 65 | M | 0 | EtOH | HCC |
| 18 | 26.8 | 1.8 | 1 | 42 | F | 0 | 4100 | |
| 19 | no | no | 1 | 59 | F | 0 | HepC | NASH |
| | read | read | | | | | | |
| 20 | 28.4 | 1.4 | 1 | 66 | F | 0 | HepC | HCC |
| 21 | 10.3 | 21.3 | 1 | 26 | M | 0 | 4100 | |
| 22 | 19.5 | 5.4 | 1 | 64 | M | 0 | NASH | HCC |
| 23 | 27.5 | 1.6 | 1 | 60 | F | 1 | NASH | |
| 24 | 25.3 | 2.2 | 1 | 44 | M | 0 | HepC | |
| 25 | 36.2 | 0.4 | 1 | 56 | M | 0 | 4403 | 4245 |
| 26 | 28.3 | 1.4 | 1 | 61 | M | 1 | 9911 | |
| 27 | 21.2 | 4.2 | 1 | 46 | M | 0 | EtOH | |
| 28 | 18.3 | 6.4 | 1 | 52 | M | 0 | 4241 | |
| 29 | 28.6 | 1.4 | 1 | 51 | F | 1 | HepC | HCC |
| 30 | 32.9 | 0.7 | 1 | 42 | M | 0 | 4241 | |
| 31 | 23.9 | 2.8 | 1 | 52 | M | 0 | 4260 | |
| 32 | 5.4 | 44.5 | 1 | 63 | M | 0 | HepC | HCC |
| 33 | 19.3 | 5.5 | 1 | 49 | M | 0 | EtOH | |
| 34 | 27.7 | 1.6 | 1 | 44 | M | 0 | HepC | |
| 35 | 19.9 | 5.1 | 1 | 63 | M | 0 | HepC | HCC |
| 36 | 20.2 | 4.8 | 1 | 50 | M | 0 | HepC | |
| 37 | 24.9 | 2.4 | 1 | 60 | M | 1 | HepC | |
| 38 | 25.8 | 2.1 | 1 | 45 | M | 1 | 4430 | |
| 39 | 24.8 | 2.4 | 1 | 71 | M | 0 | 4216 | HCC |
| 40 | 16.2 | 8.8 | 1 | 52 | M | 0 | EtOH | |
| 41 | 27.1 | 1.7 | 1 | 22 | M | 0 | 4100 | |
| 42 | 33.8 | 0.6 | 1 | 57 | F | 0 | EtOH | |
| 43 | 21.5 | 4 | 1 | 64 | M | 0 | EtOH | HCC |
| 44 | 14.8 | 10.9 | 1 | 62 | F | 0 | 4220 | HCC |
| 45 | 22.2 | 3.6 | 1 | 63 | M | 0 | HepB | |
| 46 | 22 | 3.7 | 1 | 62 | M | 0 | NASH | HCC |
| 47 | 19.6 | 5.3 | 1 | 53 | F | 1 | EtOH | |
| 48 | 23.9 | 2.8 | 1 | 68 | F | 0 | 4102 | HCC |
| 49 | 36.6 | 0.4 | 1 | 62 | M | 0 | HepC | HCC |
| 50 | 29.7 | 1.2 | 1 | 56 | M | 0 | HepC | HCC |
| 51 | 29 | 1.3 | 1 | 59 | M | 0 | 4213 | |
| 52 | 15.6 | 9.6 | 1 | 65 | M | 0 | HepC | HCC |
| 53 | 27.6 | 1.6 | 1 | 65 | M | 0 | Acute | |
| | | | | | | | Rjxn | |

TABLE 3

| | | Phys | | | | | | |
|---|---|---|---|---|---|---|---|---|
| Recipient | Race | MELD | INR | Cr | TBili | Plts | Alb | PAlb |
| 1 | White | 39 | 1.5 | 4 | 44.1 | 41 | 3 | 6 |
| 2 | White | 39 | 1.6 | 4 | 41.2 | 47 | 3.7 | 4 |
| 3 | Hispanic | 33 | 1.5 | 4 | 9 | 62 | 3.2 | 11 |
| 4 | White | 16 | 1.5 | 1 | 3.8 | 30 | 2.2 | na |
| 5 | Asian | 8 | 1.1 | 1 | 1.2 | 141 | 4 | 19.2 |
| 6 | Hispanic | 33 | 3.7 | 1 | 24 | 86 | 3.2 | 21.2 |
| 7 | Hispanic | 7 | 1.1 | 1 | 1 | 131 | 3.3 | na |
| 8 | Hispanic | 46 | 3.2 | 4 | 32.6 | 26 | 4 | 16 |
| 9 | Asian | 33 | 3.6 | 1 | 28.4 | 50 | 2.6 | 17.5 |
| 10 | Hispanic | 9 | 1.3 | 1 | 1 | 92 | 3.1 | 20.7 |
| 11 | Black | 39 | 3.2 | 2.2 | 25.1 | 76 | 3.2 | 4.2 |
| 12 | White | 19 | 1.7 | 1 | 6.1 | 59 | 2.3 | 3.3 |
| 13 | White | 7 | 1.1 | 1 | 1 | 82 | 3.4 | 25.3 |
| 14 | Other | 42 | 2.4 | 4 | 26.8 | 68 | 2.9 | na |
| 15 | White | 43 | 2.3 | 4 | 40.5 | 27 | 4 | 7.2 |
| 16 | White | 14 | 1.4 | 1 | 2.5 | 107 | 1.8 | na |
| 17 | White | 11 | 1.2 | 1 | 1.8 | 228 | 2.6 | na |
| 18 | White | 34 | 1.9 | 4 | 6.4 | 59 | 4 | 13 |
| 19 | Hispanic | 38 | 1.9 | 4 | 17.7 | 38 | 4.8 | 5 |
| 20 | Hispanic | 7 | 1.1 | 1 | 1 | 77 | 3.6 | 29 |
| 21 | White | 35 | 2.4 | 4 | 4 | 74 | 3.3 | 34 |
| 22 | White | 12 | 1.2 | 1 | 2.6 | 50 | 3.2 | 17 |
| 23 | Hispanic | 42 | 2.2 | 4 | 37.8 | 25 | 5.5 | 6 |
| 24 | Black | 30 | 2.3 | 1.5 | 14.2 | 36 | 3 | 13.2 |
| 25 | Black | 30 | 3.2 | 1 | 16.3 | 154 | 3.3 | 21.1 |
| 26 | Hispanic | 42 | 2.2 | 4 | 34 | 74 | 4.8 | 8.8 |
| 27 | White | 38 | 1.8 | 4 | 21.3 | 45 | 2.9 | 23.4 |
| 28 | White | 13 | 1.2 | 1 | 3.3 | 64 | 2.8 | 9.7 |
| 29 | White | 34 | 2 | 4 | 6.2 | 85 | 4.1 | 14.2 |
| 30 | White | 30 | 1.7 | 1.5 | 33.5 | 65 | 2.6 | 24 |
| 31 | Hispanic | 33 | 1.3 | 4 | 14.3 | 102 | 2.6 | 16.8 |
| 32 | Hispanic | 8 | 1.1 | 1 | 1.1 | 69 | 3.3 | 13 |
| 33 | Hispanic | 24 | 2.1 | 1 | 11 | 59 | 2.2 | 20.2 |
| 34 | Hispanic | 32 | 1.8 | 3 | 9.9 | 35 | 2.5 | 16.2 |
| 35 | White | 15 | 1.3 | 1.1 | 3.3 | 50 | 3 | 21.4 |
| 36 | Hispanic | 39 | 3.1 | 4 | 6.1 | 39 | 5 | 3.4 |
| 37 | Other | 42 | 2.2 | 4 | 38.3 | 57 | 3.1 | 5.1 |
| 38 | Hispanic | 9 | 1.1 | 1.2 | 1 | 224 | 4.6 | 26.7 |
| 39 | Hispanic | 8 | 1.1 | 1 | 1.1 | 57 | 3.7 | 19 |
| 40 | Hispanic | 16 | 1.4 | 1.5 | 1.6 | 74 | 3.5 | 26.3 |
| 41 | White | 42 | 3.3 | 4 | 11 | 61 | 3 | 10.5 |
| 42 | White | 32 | 2.4 | 1.7 | 15.8 | 74 | 2.4 | 17.1 |
| 43 | White | 10 | 1.3 | 1 | 1.3 | 64 | 3.3 | na |
| 44 | Hispanic | 9 | 1.1 | 1.1 | 1.2 | 62 | 3.2 | 12.5 |
| 45 | Other | 39 | 1.6 | 4 | 40.4 | 89 | 3.6 | 3.3 |
| 46 | White | 29 | 1.2 | 4 | 6.6 | 58 | 3.4 | 11.3 |
| 47 | White | 22 | 1.8 | 1.5 | 3.9 | 43 | 3.6 | 7.8 |
| 48 | Asian | 6 | 1 | 1 | 1 | 116 | 4.6 | na |
| 49 | White | 7 | 1.1 | 1 | 1 | 64 | 3.4 | 25.3 |
| 50 | Hispanic | 13 | 1.3 | 1 | 2.9 | 47 | 3.5 | na |
| 51 | White | 11 | 1.3 | 1 | 1.6 | 47 | 3.4 | 20 |
| 52 | Black | 8 | 1.2 | 1 | 1 | 144 | 3.9 | 12.1 |
| 53 | Other | 43 | 2.1 | 4 | 55.2 | 102 | 3.2 | 24.2 |

TABLE 4

| Recipient | Pressors | HD | Vent | Redo | #OLT | L/K | HT | WT | BMI |
|---|---|---|---|---|---|---|---|---|---|
| 1 | 0 | 1 | 0 | 1 | 4 | 1 | 152.4 | 53.1 | 22.86 |
| 2 | 0 | 1 | 0 | 1 | 4 | 1 | 152.4 | 53.1 | 22.86 |
| 3 | 0 | 1 | 0 | 1 | 3 | 0 | 167.64 | 62 | 22.06 |
| 4 | 0 | 0 | 0 | 0 | 1 | 0 | 188 | 96 | 27.16 |
| 5 | 0 | 0 | 0 | 0 | 1 | 0 | 162.56 | 65 | 24.60 |
| 6 | 0 | 0 | 0 | 0 | 1 | 0 | 162.56 | 100 | 37.84 |
| 7 | 0 | 0 | 0 | 0 | 1 | 0 | 152.4 | 70.91 | 30.53 |
| 8 | 2 | 1 | 1 | 0 | 1 | 0 | | | |
| 9 | 0 | 0 | 0 | 0 | 1 | 0 | 170.18 | 97.27 | 33.59 |
| 10 | 0 | 0 | 0 | 0 | 1 | 0 | 160.02 | 75 | 29.29 |
| 11 | 0 | 0 | 0 | 0 | 1 | 0 | 165.1 | 56.7 | 20.80 |
| 12 | 0 | 0 | 0 | 0 | 1 | 0 | | | |
| 13 | 0 | 0 | 0 | 0 | 1 | 0 | 180.34 | 90.73 | 27.90 |
| 14 | 0 | 0 | 0 | 0 | 1 | 0 | 170.18 | 60 | 20.72 |
| 15 | 0 | 1 | 0 | 1 | 2 | 1 | 190.5 | 76 | 20.94 |
| 16 | 0 | 0 | 0 | 0 | 1 | 0 | 175.26 | 76.36 | 24.86 |
| 17 | 0 | 0 | 0 | 0 | 1 | 0 | 203.2 | 109.09 | 26.42 |
| 18 | 0 | 1 | 1 | 0 | 1 | 0 | 154.94 | 76.32 | 31.79 |
| 19 | 1 | 1 | 1 | 0 | 1 | 0 | 167.64 | 110.45 | 39.30 |
| 20 | 0 | 0 | 0 | 0 | 1 | 0 | 160.02 | 94.09 | 36.75 |
| 21 | 1 | 1 | 1 | 0 | 1 | 0 | 177.8 | 85 | 26.89 |
| 22 | 0 | 0 | 0 | 0 | 1 | 0 | 172.72 | 98.18 | 32.91 |
| 23 | 3 | 1 | 1 | 0 | 1 | 0 | 160.782 | 67.64 | 26.16 |
| 24 | 0 | 0 | 0 | 0 | 1 | 0 | 185.42 | 79.55 | 23.14 |
| 25 | 0 | 0 | 0 | 0 | 1 | 0 | 180.34 | 75 | 23.06 |
| 26 | 1 | 1 | 1 | 1 | 2 | 0 | 167.64 | 83 | 29.53 |
| 27 | 1 | 1 | 1 | 0 | 1 | 0 | 177.8 | 68.4 | 21.64 |
| 28 | 0 | 0 | 0 | 0 | 1 | 0 | 177.8 | 84.09 | 26.60 |
| 29 | 0 | 1 | 0 | 0 | 1 | 0 | 152.4 | 56.7 | 24.41 |
| 30 | 0 | 0 | 0 | 0 | 1 | 0 | 180.34 | 79 | 24.29 |
| 31 | 1 | 1 | 1 | 1 | 2 | 1 | 165.1 | 68 | 24.95 |
| 32 | 0 | 0 | 0 | 0 | 1 | 0 | 182.88 | 95 | 28.40 |
| 33 | 0 | 0 | 0 | 0 | 1 | 0 | 177.8 | 110.45 | 34.94 |
| 34 | 0 | 0 | 0 | 0 | 1 | 0 | 170.18 | 117.27 | 40.49 |
| 35 | 0 | 0 | 0 | 0 | 1 | 0 | 187.96 | 74.27 | 21.02 |
| 36 | 0 | 1 | 0 | 0 | 1 | 0 | 167.6 | 79.4 | 28.27 |
| 37 | 0 | 1 | 0 | 0 | 1 | 0 | 167.6 | 71.2 | 25.34 |
| 38 | 0 | 0 | 0 | 0 | 1 | 0 | 175.26 | 112.27 | 36.55 |
| 39 | 0 | 0 | 0 | 0 | 1 | 0 | 162.56 | 69.45 | 26.28 |
| 40 | 0 | 0 | 0 | 0 | 1 | 0 | 165.1 | 80.45 | 29.52 |
| 41 | 0 | 1 | 1 | 0 | 1 | 0 | 172.72 | 68.4 | 22.92 |
| 42 | 0 | 0 | 0 | 0 | 1 | 0 | 167.64 | 72.91 | 25.94 |
| 43 | 0 | 0 | 0 | 0 | 1 | 0 | 162.56 | 83.64 | 31.65 |
| 44 | 0 | 0 | 0 | 0 | 1 | 0 | 154.94 | 78.27 | 32.60 |
| 45 | 0 | 1 | 1 | 0 | 1 | 0 | 162.56 | 72.27 | 27.35 |
| 46 | 0 | 1 | 0 | 0 | 1 | 1 | 182.88 | 110.45 | 33.03 |
| 47 | 0 | 0 | 0 | 0 | 1 | 0 | 162.6 | 66 | 24.96 |
| 48 | 0 | 0 | 0 | 0 | 1 | 0 | 157.48 | 71.81 | 28.96 |
| 49 | 0 | 0 | 0 | 0 | 1 | 0 | 180.34 | 90.72 | 27.90 |
| 50 | 0 | 0 | 0 | 0 | 1 | 0 | 165.1 | 64.64 | 23.71 |
| 51 | 0 | 0 | 0 | 0 | 1 | 0 | 170.18 | 81.82 | 28.25 |
| 52 | 0 | 0 | 0 | 0 | 1 | 0 | 180.34 | 88.64 | 27.25 |
| 53 | 0 | 1 | 0 | 1 | 2 | 1 | 172.7 | 65.6 | 21.99 |

TABLE 5

| | | | 7 d | 30 d | 6 m | 1 yr | Rpfn | LD |
|---|---|---|---|---|---|---|---|---|
| Recip | Takeback# | ReOLT | surv | surv | surv | surv | Bx | 1 d |
| 1 | na | na | na | na | na | na | na | na |
| 2 | na | na | na | na | na | na | na | na |
| 3 | na | na | na | na | na | na | na | na |
| 4 | na | na | na | na | na | na | na | na |
| 5 | na | na | na | na | na | na | na | na |
| 6 | na | na | na | na | na | na | na | na |
| 7 | na | na | na | na | na | na | na | na |
| 8 | na | na | na | na | na | na | na | na |
| 9 | na | na | na | na | na | na | na | na |
| 10 | na | na | na | na | na | na | na | na |
| 11 | na | na | na | na | na | na | na | na |
| 12 | na | na | na | na | na | na | na | na |
| 13 | na | na | na | na | na | na | na | na |
| 14 | na | na | na | na | na | na | na | na |
| 15 | na | na | na | na | na | na | na | na |
| 16 | 0 | 0 | 1 | 1 | 1 | 1 | Min IR, | 969 |
| | | | | | | | Min St | |
| 17 | 1 | 0 | 1 | 1 | 1 | 1 | Mod IRI | 2645 |
| 18 | 2 | 0 | 1 | 1 | 1 | ? | mild ir | 2170 |
| | | | | | | | mild st 20 | |
| 19 | 0 | 0 | 1 | 1 | 1 | ? | Min IR, | 169 |
| | | | | | | | no st | |
| 20 | 0 | 0 | 1 | 1 | 1 | 1 | Min IR, | 550 |
| | | | | | | | min st | |
| 21 | 0 | 0 | 1 | 1 | 1 | 1 | 0 | 1837 |
| 22 | 0 | 0 | 1 | 1 | 1 | ? | Min IR | 337 |
| 23 | 3 | 0 | 1 | 1 | 0 | 0 | mild ir | 585 |
| 24 | 0 | 0 | 1 | 1 | 1 | 1 | min IR | 535 |
| | | | | | | | mld St | |
| 25 | 0 | 0 | 1 | 1 | 1 | 1 | mod IR | 412 |
| | | | | | | | min st | |
| 26 | 1 | 0 | 0 | 0 | 0 | 0 | Sev nec, | 5132 |
| | | | | | | | no st, | |
| | | | | | | | no fib | |
| 27 | 0 | 0 | 1 | 1 | 1 | 1 | mild ir | 904 |
| | | | | | | | mild st 30 | |
| 28 | 1 (wound) | 0 | 1 | 1 | 1 | 1 | Min IR | 223 |
| 29 | 4 | 0 | 1 | 1 | 0 | 0 | mild st | 1395 |
| | | | | | | | 15-20 | |
| | | | | | | | md iri | |
| 30 | 0 | 0 | 1 | 1 | 1 | 1 | No macro, | 202 |
| | | | | | | | 80% micro | |
| 31 | 0 | 0 | 1 | 1 | 1 | ? | min ir | 198 |
| | | | | | | | no st | |
| 32 | 0 | 0 | 1 | 1 | 1 | ? | no st | 378 |
| | | | | | | | min IR | |
| 33 | 0 | 0 | 1 | 1 | ? | na | 0 | 204 |
| 34 | 0 | 0 | 1 | 1 | 1 | m | no st | 298 |
| 35 | 0 | 0 | 1 | 1 | ? | na | mild st 10 | 1156 |
| 36 | 0 | 0 | 1 | 1 | 1 | na | mild st | 431 |
| 37 | 0 | 0 | 0 | 0 | 0 | 0 | min st | na |
| 38 | 4 | 1 | 1 | 0 | 0 | 0 | min St | 5060 |
| | | | | | | | min IR | |
| 39 | 1 | 0 | 1 | 1 | 1 | na | min St | 319 |
| | | | | | | | min IR | |
| 40 | 0 | 0 | 1 | 1 | na | na | mild ir | 359 |
| | | | | | | | min st 10 | |
| 41 | 2 | 0 | 1 | 1 | 1 | na | Min IR, | 1141 |
| | | | | | | | min st | |
| 42 | 1 | 0 | 1 | 1 | na | na | min ir | 238 |
| | | | | | | | min st | |
| 43 | 0 | 0 | 1 | 1 | n | na | min IR | 429 |
| 44 | 1 | 0 | 1 | 1 | na | na | min IR | 331 |
| 45 | 0 | 0 | 1 | 1 | na | na | min ir | 175 |
| 46 | 2 | 0 | 1 | 1 | na | na | min IR | 221 |
| 47 | 0 | 0 | 1 | 1 | 0 | 0 | mild st | 1158 |
| | | | | | | | 20 min | |
| | | | | | | | ir fib | |
| 48 | 0 | 0 | 1 | 1 | na | na | na | 440 |
| 49 | 1 | 0 | 1 | 1 | na | na | mild ir | 2295 |
| 50 | 0 | 0 | 1 | 1 | na | na | 0 | 435 |
| 51 | 0 | 0 | 1 | 1 | na | na | mild st | 2560 |
| | | | | | | | 20 mod ir | |
| 52 | 0 | 0 | 1 | 1 | na | na | no st | 3984 |
| | | | | | | | mod IR | |
| 53 | 1 wnd | 0 | 1 | 1 | na | na | mild ir | 303 |

TABLE 6

| | AST | ALT | Tbili | INR | LD | AST | ALT | Tbili |
|---|---|---|---|---|---|---|---|---|
| Recipient | 1 d | 1 d | 1 d | 1 d | 7 d | 7 d | 7 d | 7 d |
| 1 | na | na | na | na | na | na | na | na |
| 2 | na | na | na | na | na | na | na | na |
| 3 | na | na | na | na | na | na | na | na |
| 4 | na | na | na | na | na | na | na | na |
| 5 | na | na | na | na | na | na | na | na |
| 6 | na | na | na | na | na | na | na | na |
| 7 | na | na | na | na | na | na | na | na |
| 8 | na | na | na | na | na | na | na | na |
| 9 | na | na | na | na | na | na | na | na |
| 10 | na | na | na | na | na | na | na | na |
| 11 | na | na | na | na | na | na | na | na |
| 12 | na | na | na | na | na | na | na | na |
| 13 | na | na | na | na | na | na | na | na |
| 14 | na | na | na | na | na | na | na | na |
| 15 | na | na | na | na | na | na | na | na |
| 16 | 742 | 374 | 4 | 1.1 | 266 | 37 | 127 | 1.8 |
| 17 | 2463 | 1018 | 4 | 1.5 | 250 | 46 | 311 | 2.8 |
| 18 | 1579 | 1957 | 3.9 | 1.1 | 279 | 45 | 186 | 5.7 |
| 19 | 100 | 119 | 3.7 | 1.1 | 154 | 14 | 38 | 1.8 |
| 20 | 273 | 327 | 2.6 | 1.2 | 468 | 117 | 411 | 1.2 |
| 21 | 4087 | 2688 | 1.5 | 1.1 | 323 | 43 | 341 | 1.7 |
| 22 | 669 | 561 | 3.4 | 1.2 | 190 | 36 | 157 | 1.5 |
| 23 | 791 | 463 | 14.7 | 1.3 | 354 | 45 | 126 | 28.8 |
| 24 | 530 | 360 | 2.3 | 1.2 | 413 | 49 | 108 | 2.8 |
| 25 | 681 | 672 | 5.7 | 1.3 | 575 | 53 | 204 | 3.6 |
| 26 | 6536 | 1985 | 5.3 | 1.5 | na | na | na | na |
| 27 | 1191 | 505 | 7.1 | 1.6 | 239 | 23 | 63 | 4.3 |
| 28 | 176 | 193 | 3.2 | 1.2 | 173 | 17 | 49 | 1.9 |
| 29 | 1642 | 1126 | 1.4 | 2.2 | 314 | 37 | 106 | 2.3 |
| 30 | 268 | 406 | 10.3 | 1.4 | 188 | 21 | 77 | 9.1 |
| 31 | 176 | 143 | 4.4 | 1.3 | 361 | 30 | 40 | 4.2 |
| 32 | 854 | 477 | 4 | 1.4 | 236 | 38 | 102 | 4.3 |
| 33 | 138 | 86 | 5.6 | 1.2 | 194 | 26 | 63 | 3.1 |
| 34 | 307 | 186 | 6.9 | 1.2 | 400 | 270 | 433 | 5.7 |
| 35 | 2374 | 1451 | 5.6 | 1.5 | 631 | 1605 | 1136 | 3.8 |
| 36 | 431 | 259 | 5.1 | 1.3 | 204 | 27 | 57 | 2.8 |
| 37 | na | na | na | na | na | na | na | na |
| 38 | 3385 | 2010 | 3.1 | 1.3 | 259 | 20 | 133 | 1.2 |
| 39 | 181 | 189 | 1.8 | 1.2 | 229 | 15 | 37 | 0.6 |
| 40 | 485 | 356 | 3.8 | 1.3 | 201 | 33 | 113 | 1.1 |
| 41 | 851 | 1546 | 10.5 | 1.4 | 399 | 59 | 379 | 3.1 |
| 42 | 344 | 527 | 3.4 | 1.2 | 190 | 20 | 59 | 5.8 |
| 43 | 839 | 846 | 0.9 | 1.3 | 251 | 25 | 104 | 0.6 |
| 44 | 268 | 256 | 22 | 1.1 | 549 | 114 | 136 | 2.7 |
| 45 | 89 | 179 | 6.4 | 1.2 | 363 | 15 | 31 | 3.5 |
| 46 | 347 | 141 | 5.1 | 1.5 | 157 | 11 | 20 | 1.4 |
| 47 | 992 | 296 | 4.3 | 1.5 | 240 | 39 | 62 | 4.7 |
| 48 | 293 | 406 | 0.6 | 1 | 349 | 38 | 111 | 0.6 |
| 49 | 1730 | 1112 | 2 | 1.5 | 306 | 59 | 204 | 2.5 |
| 50 | 466 | 523 | 2.3 | 1.3 | 382 | 55 | 147 | 1.4 |
| 51 | 3948 | 1645 | 3.4 | 1.3 | 286 | 43 | 266 | 1.7 |
| 52 | 3792 | 1505 | 3.1 | 1.5 | 191 | 67 | 202 | 1.5 |
| 53 | 197 | 103 | 20.8 | 1.1 | 257 | 26 | 75 | 16.3 |

TABLE 7

| | INR | LD | AST | ALT | Tbili | INR |
|---|---|---|---|---|---|---|
| Recip | 7 d | 30 d | 30 d | 30 d | 30 d | 30 d |
| 1 | na | na | na | na | na | na |
| 2 | na | na | na | na | na | na |
| 3 | na | na | na | na | na | na |
| 4 | na | na | na | na | na | na |
| 5 | na | na | na | na | na | na |
| 6 | na | na | na | na | na | na |
| 7 | na | na | na | na | na | na |
| 8 | na | na | na | na | na | na |
| 9 | na | na | na | na | na | na |
| 10 | na | na | na | na | na | na |
| 11 | na | na | na | na | na | na |
| 12 | na | na | na | na | na | na |
| 13 | na | na | na | na | na | na |
| 14 | na | na | na | na | na | na |
| 15 | na | na | na | na | na | na |
| 16 | 1.1 | na | 42 | 79 | 0.6 | 1.1 |
| 17 | 1.1 | 231 | 36 | 85 | 0.9 | 1 |
| 18 | 1.1 | 309 | 15 | 15 | 1.2 | 2.6 |
| 19 | 1 | 195 | 9 | 8 | 1.1 | 1.1 |
| 20 | 1.2 | 255 | 57 | 163 | 0.8 | 1.1 |
| 21 | 1.5 | 313 | 22 | 28 | 0.8 | 1.1 |
| 22 | 1.2 | 142 | 12 | 20 | 0.5 | 1.1 |
| 23 | 1.2 | 388 | 101 | 236 | 7.4 | 1.1 |
| 24 | 1.1 | 248 | 13 | 17 | 0.7 | 1 |
| 25 | 1.1 | 177 | 21 | 21 | 1.6 | 1.1 |
| 26 | na | na | na | na | na | na |
| 27 | 1.1 | 136 | 14 | 7 | 0.9 | 1.2 |
| 28 | 1.3 | na | 12 | 12 | 0.8 | 1.2 |
| 29 | 1.3 | 298 | 113 | 90 | 18.4 | 1.2 |
| 30 | 1.3 | 120 | 17 | 43 | 2 | 1.1 |
| 31 | 1.1 | 266 | 16 | 14 | 1.5 | 1.1 |
| 32 | 1.2 | 165 | 34 | 26 | 1.2 | 1 |
| 33 | 1.1 | na | 16 | 13 | 1.8 | na |
| 34 | 1.2 | 186 | 144 | 354 | 1.4 | 1.5 |
| 35 | 1.3 | na | 23 | 25 | 1.2 | na |
| 36 | 1.2 | 218 | 12 | 8 | 0.9 | 1.1 |
| 37 | na | na | na | na | na | na |
| 38 | 1.3 | na | na | na | na | na |
| 39 | 1.2 | 160 | 9 | 7 | 0.5 | 1.1 |
| 40 | 1.2 | na | 8 | 10 | 0.7 | na |
| 41 | 1.1 | 215 | 24 | 40 | 1.1 | 1.2 |
| 42 | 1.1 | 221 | 12 | 11 | 0.9 | 1 |
| 43 | 1.2 | na | 13 | 16 | 0.4 | na |
| 44 | 1.1 | 468 | 28 | 19 | 0.9 | 1 |
| 45 | 1.1 | 178 | 15 | 5 | 1.8 | 1 |
| 46 | 1.3 | na | 12 | 10 | 0.8 | 1.1 |
| 47 | 1.2 | 128 | 16 | 9 | 1 | 1.2 |
| 48 | 1.1 | 305 | 42 | 76 | 0.5 | 1 |
| 49 | 1.2 | 253 | 129 | 328 | 0.6 | 1 |
| 50 | 1.2 | na | 26 | 35 | 0.9 | na |
| 51 | 1.2 | 195 | 17 | 38 | 0.6 | 1.1 |
| 52 | 1.1 | na | 32 | 43 | 0.5 | na |
| 53 | 1.3 | 202 | 9 | 8 | 1.3 | 1.1 |

TABLE 8

| | | | | | Wt | Ht |
|---|---|---|---|---|---|---|
| Donors | Age | Male | Race | ABO | (kg) | (cm) |
| 1 | 24 | 1 | Latino | A | 55 | 167.64 |
| 2 | 29 | 0 | Latino | A | 73 | 165.1 |
| 3 | 39 | 1 | White | O | 80 | 172.72 |
| 4 | 49 | 1 | White | O | 80 | 177.8 |
| 5 | 68 | 1 | Asian | O | 41 | 162.56 |
| 6 | 53 | 0 | Latino | O | 80 | 160.02 |
| 7 | 55 | 0 | Latino | O | 60 | 167.64 |
| 8 | 0 | 1 | Asian | O | 70 | 180.34 |
| 9 | 25 | 0 | Black | A | 80 | 162.56 |
| 10 | 65 | 1 | White | B | 73.5 | 170.18 |
| 11 | 57 | 0 | White | A | 49.2 | 160.02 |
| 12 | 30 | 1 | White | O | 120 | 188 |
| 13 | 59 | 1 | White | A | 102.5 | 180.34 |
| 14 | 46 | 1 | White | O | 80 | 170.18 |
| 15 | 50 | 0 | White | O | 65 | 167 |
| 16 | 22 | 1 | White | O | 85 | 180.34 |
| 17 | 54 | 1 | White | A | 115 | 187.96 |
| 18 | 43 | 1 | Latino | A | 77 | 172.72 |
| 19 | 20 | 1 | Black | O | 91.8 | 190.5 |
| 20 | 47 | 1 | Latino | O | 69.3 | 160.02 |
| 21 | 51 | 0 | Latino | O | 84 | 165.1 |
| 22 | 55 | 1 | White | A | 114 | 180.34 |
| 23 | 62 | 1 | Asian | A | 72 | 162.56 |
| 24 | 57 | 1 | Latino | O | 81.8 | 172.72 |
| 25 | 65 | 1 | White | O | 102 | 180.34 |
| 26 | 26 | 1 | Asian | A | 80 | 177 |
| 27 | 52 | 0 | Latino | O | 66 | 154.94 |
| 28 | 20 | 0 | Black | B | 81.8 | 182.88 |
| 29 | 45 | 0 | Latino | A | 86 | 170.18 |
| 30 | 22 | 1 | White | B | 73 | 177.8 |
| 31 | 41 | 1 | White | A | 70 | 180.34 |
| 32 | 67 | 1 | Latino | A | 80 | 170.18 |
| 33 | 32 | 0 | Latino | B | 65 | 165.1 |
| 34 | 22 | 1 | Latino | A | 71 | 170.18 |
| 35 | 69 | 0 | Black | O | 137 | 175.26 |
| 36 | 48 | 1 | Latino | O | 50 | 172.72 |
| 37 | 43 | 1 | Latino | A | 73 | 162.56 |
| 38 | 44 | 1 | White | A | 80 | 183 |
| 39 | 59 | 0 | Latino | A | 81 | 160.02 |
| 40 | 65 | 1 | White | AB | 85 | 185.42 |
| 41 | 49 | 1 | Latino | A | 84 | 167 |
| 42 | 17 | 1 | Latino | B | 83.6 | 182.88 |
| 43 | 23 | 1 | Latino | O | 79.4 | 170.18 |
| 44 | 61 | 1 | Latino | O | 66 | 157.48 |
| 45 | 51 | 1 | Black | O | 118 | 165.1 |
| 46 | 36 | 0 | Latino | B | 91.7 | 162.56 |
| 47 | 54 | 1 | Latino | O | 59.6 | 162 |
| 48 | 22 | 1 | Asian | B | 78 | 168 |
| 49 | 23 | 1 | Latino | A | 95 | 175 |
| 50 | 64 | 1 | White | AB | 69.1 | 162.56 |
| 51 | 25 | 1 | White | A | 77 | 170.18 |
| 52 | 60 | 1 | Latino | O | 87 | 161 |
| 53 | 26 | 1 | White | A | 64.6 | 175.26 |

TABLE 9

| | | Cause of | | Liver | |
|---|---|---|---|---|---|
| Donor | BMI | Death | LOH | Trauma | Dopa |
| 1 | 19.6 | ICH | 4 | 0 | 1 |
| 2 | 26.8 | ICH | 6 | 0 | 1 |
| 3 | 26.8 | Anoxia | 4 | 0 | 1 |
| 4 | 25.3 | ICH | 4 | 0 | 1 |
| 5 | 15.5 | ICH | 3 | 0 | 1 |
| 6 | 31.2 | ICH | 125 | 0 | 0 |
| 7 | 21.3 | ICH | 3 | 0 | 0 |
| 8 | 21.5 | ICH | 4 | 0 | 0 |
| 9 | 30.3 | ICH | 17 | 0 | 0 |
| 10 | 25.4 | ICH | 7 | 0 | 0 |
| 11 | 19.2 | ICH | 3 | 0 | 0 |
| 12 | 34.0 | Blunt Head | 20 | 1 | 0 |
| | | Trauma | | | |
| 13 | 31.5 | Blunt Head | 4 | 1 | 1 |
| | | Trauma | | | |
| 14 | 27.6 | Blunt Head | 5 | 1 | 0 |
| | | Trauma | | | |
| 15 | 23.3 | ICH | 5 | 0 | 0 |
| 16 | 26.1 | Penetrating | 4 | 0 | 1 |
| | | head injury | | | |
| 17 | 32.6 | ICH | 33 | 0 | 1 |
| 18 | 25.8 | Penetrating | 5 | 0 | 0 |
| | | head injury | | | |
| 19 | 25.3 | Blunt Head | 8 | 1 | 1 |
| | | Trauma | | | |
| 20 | 27.1 | Penetrating | 6 | 0 | 1 |
| | | head injury | | | |
| 21 | 30.8 | ICH | 4 | 0 | 1 |
| 22 | 35.1 | Anoxia | 6 | 0 | 1 |
| 23 | 27.2 | ICH | 3 | 0 | 0 |
| 24 | 27.4 | ICH | 4 | 0 | 1 |
| 25 | 31.4 | ICH | 2 | 0 | 0 |
| 26 | 25.5 | Penetrating | 4 | 0 | 0 |
| | | head injury | | | |
| 27 | 27.5 | ICH | 6 | 0 | 0 |
| 28 | 24.5 | Blunt Head | 2 | 1 | 0 |
| | | Trauma | | | |
| 29 | 29.7 | ICH | 4 | 0 | 0 |
| 30 | 23.1 | Blunt Head | 4 | 1 | 0 |
| | | Trauma | | | |
| 31 | 21.5 | Blunt Head | 4 | 1 | 0 |
| | | Trauma | | | |
| 32 | 27.6 | ICH | 4 | 0 | 0 |
| 33 | 23.8 | ICH | 5 | 0 | 0 |
| 34 | 24.5 | Anoxia | 7 | 0 | 0 |
| 35 | 44.6 | ICH | 4 | 0 | 0 |
| 36 | 16.8 | ICH | 4 | 0 | 0 |
| 37 | 27.6 | Anoxia | 14 | 0 | 0 |
| 38 | 23.9 | ICH | 8 | 0 | 0 |
| 39 | 31.6 | ICH | 3 | 0 | 0 |
| 40 | 24.7 | ICH | 6 | 0 | 1 |
| 41 | 30.1 | ICH | 22 | 0 | 0 |
| 42 | 25.0 | Penetrating | 8 | 0 | 0 |
| | | head injury | | | |
| | | (GSW | | | |
| | | homicide) | | | |
| 43 | 27.4 | Blunt Head | 10 | 1 | 0 |
| | | Trauma | | | |
| 44 | 26.6 | ICH | 3 | 0 | 0 |
| 45 | 43.3 | ICH | 6 | 0 | 1 |
| 46 | 34.7 | Anoxia | 7 | 0 | 0 |
| 47 | 22.7 | Blunt Head | 8 | 1 | 0 |
| | | Trauma | | | |
| 48 | 27.6 | Blunt Head | 5 | 1 | 0 |
| | | Trauma | | | |
| 49 | 31.0 | Penetrating | 3 | 0 | 0 |
| | | head injury | | | |
| 50 | 26.1 | Anoxia | 8 | 0 | 0 |
| | | (suicide) | | | |
| 51 | 26.6 | GSW | 3 | 0 | 0 |
| | | (homicide) | | | |
| 52 | 33.6 | ICH | 5 | 0 | 1 |
| 53 | 21.0 | Blunt Head | 3 | 1 | 0 |
| | | Trauma | | | |

TABLE 10

| | | | | Total | | | |
|---|---|---|---|---|---|---|---|
| Donor | Vaso | NE | Phenyl | Pressors | Insulin | DM | HTN |
| 1 | 1 | 0 | 0 | 2 | 1 | 0 | 0 |
| 2 | 1 | 0 | 0 | 2 | 1 | 0 | 0 |
| 3 | 1 | 0 | 0 | 2 | 1 | 0 | 0 |
| 4 | 1 | 0 | 0 | 2 | 0 | 0 | 0 |
| 5 | 1 | 1 | 1 | 4 | 1 | 0 | 1 |
| 6 | 1 | 0 | 1 | 2 | 1 | 0 | 0 |
| 7 | 0 | 1 | 0 | 1 | 1 | 1 | 1 |
| 8 | 1 | 1 | 0 | 2 | 1 | 0 | 0 |
| 9 | 1 | 1 | 0 | 2 | 1 | 0 | 0 |
| 10 | 1 | 1 | 0 | 2 | 0 | 0 | 0 |
| 11 | 1 | 1 | 0 | 2 | 0 | 0 | 0 |
| 12 | 1 | 0 | 0 | 1 | 1 | 0 | 0 |
| 13 | 1 | 0 | 0 | 2 | 1 | 0 | 1 |
| 14 | 1 | 1 | 0 | 2 | 1 | 0 | 0 |
| 15 | 1 | 1 | 0 | 2 | 1 | 0 | 1 |
| 16 | 0 | 0 | 0 | 1 | 1 | 0 | 0 |
| 17 | 0 | 0 | 1 | 2 | 0 | 0 | 1 |
| 18 | 0 | 0 | 0 | 0 | 1 | 0 | 0 |
| 19 | 1 | 0 | 0 | 2 | 1 | 0 | 0 |
| 20 | 1 | 0 | 0 | 2 | 1 | 0 | 0 |
| 21 | 0 | 0 | 0 | 1 | 1 | 0 | 0 |
| 22 | 1 | 0 | 0 | 2 | 0 | 0 | 0 |
| 23 | 1 | 1 | 0 | 2 | 0 | 0 | 0 |
| 24 | 1 | 0 | 0 | 2 | 1 | 0 | 1 |
| 25 | 1 | 1 | 0 | 2 | 0 | 0 | 0 |
| 26 | 1 | 1 | 0 | 2 | 1 | 0 | 0 |
| 27 | 0 | 0 | 0 | 0 | 1 | 1 | 1 |
| 28 | 1 | 0 | 0 | 1 | 1 | 0 | 0 |
| 29 | 1 | 1 | 0 | 2 | 1 | 0 | 0 |
| 30 | 1 | 1 | 1 | 3 | 0 | 0 | 0 |
| 31 | 0 | 1 | 0 | 1 | 0 | 0 | 0 |
| 32 | 1 | 1 | 0 | 2 | 0 | 0 | 1 |
| 33 | 1 | 1 | 0 | 2 | 1 | 0 | 0 |
| 34 | 1 | 0 | 0 | 1 | 1 | 0 | 0 |
| 35 | 1 | 1 | 0 | 2 | 1 | 0 | 1 |
| 36 | 1 | 0 | 1 | 2 | 0 | 0 | 0 |
| 37 | 1 | 0 | 0 | 1 | 1 | 0 | 0 |
| 38 | 1 | 1 | 0 | 2 | 0 | 0 | 1 |
| 39 | 1 | 1 | 0 | 2 | 0 | 0 | 1 |
| 40 | 1 | 0 | 0 | 2 | 1 | 0 | 1 |
| 41 | 1 | 1 | 0 | 2 | 1 | 0 | 0 |
| 42 | 1 | 0 | 1 | 2 | 0 | 0 | 0 |
| 43 | 1 | 0 | 0 | 1 | 0 | 0 | 0 |
| 44 | 1 | 0 | 0 | 1 | 1 | 1 | 1 |
| 45 | 1 | 1 | 0 | 3 | 0 | 0 | 1 |
| 46 | 1 | 1 | 0 | 2 | 0 | 0 | 0 |
| 47 | 0 | 1 | 0 | 1 | 1 | 0 | 0 |
| 48 | 1 | 0 | 0 | 1 | 1 | 0 | 0 |
| 49 | 1 | 1 | 0 | 2 | 1 | 0 | 0 |
| 50 | 0 | 1 | 0 | 1 | 1 | 1 | 1 |
| 51 | 1 | 0 | 0 | 1 | 1 | 0 | 1 |
| 52 | 0 | 1 | 0 | 2 | 1 | 1 | 1 |
| 53 | 0 | 0 | 1 | 1 | 0 | 0 | 0 |

TABLE 11

| | EtOH | | | HepB | |
|---|---|---|---|---|---|
| Donor | Abuse | Other Drugs | HgbA1c | Core | HepC |
| 1 | 0 | none | 5.8 | 0 | 0 |
| 2 | 0 | none | 5.1 | 0 | 0 |
| 3 | 0 | opiates | 5.8 | 0 | 0 |
| 4 | unk | IVDA, meth, | 5.8 | 0 | 1 |
| | | heroine, | | | |
| | | marijuana | | | |
| 5 | 1 | none | not done | 0 | 0 |
| 6 | 0 | none | 6.0 | 0 | 0 |
| 7 | 0 | none | 8.9 | 0 | 0 |
| 8 | 0 | none | 5.3 | 0 | 0 |
| 9 | 0 | meth, | 5.5 | 1 | 0 |
| | | marijuana | | | |
| 10 | 0 | none | not done | 0 | 0 |
| 11 | 1 | marijuana | not done | 0 | 0 |
| 12 | 0 | marijuana | 5.6 | 0 | 0 |
| 13 | 0 | marijuana | not done | 0 | 0 |
| 14 | 1 | mar, benzos | 4.8 | 0 | 0 |
| 15 | 0 | amphetamine | 5.5 | 0 | 0 |
| 16 | 0 | marijuana | 5.2 | 0 | 0 |
| 17 | 0 | none | 5.1 | 0 | 0 |
| 18 | 0 | none | 5.5 | 0 | 0 |
| 19 | 0 | amphetamine, | 5.5 | 0 | 0 |
| | | THC | | | |
| 20 | 1 | cocaine, meth | 6.3 | 0 | 0 |
| 21 | 0 | none | 9.6 | 0 | 0 |
| 22 | 0 | crack | 6.8 | 0 | 0 |
| 23 | 0 | none | not done | 0 | 0 |
| 24 | 1 | opiates, | not done | 0 | 1 |
| | | marijuana | | | |
| 25 | 0 | none | not done | 0 | 0 |
| 26 | 0 | none | 5.3 | 0 | 0 |
| 27 | 0 | none | 13.9 | 0 | 0 |
| 28 | 0 | none | 5.5 | 0 | 0 |
| 29 | 0 | none | 6.3 | 0 | 0 |
| 30 | 0 | none | 5.1 | 0 | 0 |
| 31 | 0 | IVDA, meth, | 5.7 | 0 | 0 |
| | | prescript Rx | | | |
| 32 | unk | none | not done | 0 | 0 |
| 33 | 0 | cocaine, | 5.2 | 0 | 0 |
| | | opiates | | | |
| 34 | 0 | cocaine, | not | 0 | 0 |
| | | marijuana | done | | |
| 35 | 0 | none | not | 0 | 0 |
| | | | done | | |
| 36 | 1 | none | 6.0 | 0 | 0 |
| 37 | 0 | none | 6.1 | 0 | 0 |
| 38 | 1 | none | 5.0 | 0 | 0 |
| 39 | 0 | none | not | 0 | 0 |
| | | | done | | |
| 40 | 0 | none | 6.0 | 0 | 0 |
| 41 | 0 | none | 6.4 | 0 | 0 |
| 42 | 0 | none | 5.4 | 0 | 0 |
| 43 | 0 | marijuana | 5.6 | 0 | 0 |
| 44 | 0 | none | not | 0 | 0 |
| | | | done | | |
| 45 | 0 | none | 5.7 | 0 | 0 |
| 46 | 0 | none | 5.1 | 0 | 0 |
| 47 | 1 | none | 5.8 | 0 | 0 |
| 48 | 0 | marijuana, | 5.3 | 0 | 0 |
| | | ecstasy, | | | |
| | | amphetamine | | | |
| 49 | 0 | amphetamines | not | 0 | 0 |
| | | | done | | |
| 50 | 0 | none | not | 0 | 0 |
| | | | done | | |
| 51 | 0 | marijuana, | 5.8 | 0 | 0 |
| | | ecstasy, | | | |
| | | amphetamine | | | |
| 52 | 0 | none | not | 0 | 0 |
| | | | done | | |
| 53 | 0 | meth, benzo, | 4.8 | 0 | 0 |
| | | marijuana | | | |

TABLE 12

| | Cardiac | Respir | Total | CPR | |
|---|---|---|---|---|---|
| Donor | Arrest | Arrest | Downtime | Duration | MAP range |
| 1 | 1 | 1 | 8 | 8 | 64.3-136 |
| 2 | 0 | 0 | 0 | 0 | 69.7-107.7 |
| 3 | 1 | 1 | 32 | 32 | 69.3-119.7 |
| 4 | 0 | 0 | 0 | 0 | 72.3-123 |
| 5 | 1 | 0 | unk | 0 | 57.7-96 |
| 6 | 0 | 1 | 21 | 21 | 66.3-125 |
| 7 | 0 | 0 | 0 | 0 | 85.3-110.7 |
| 8 | 0 | 0 | 0 | 0 | 73.3-148.3 |
| 9 | 0 | 0 | 0 | 0 | 63.7-135.7 |
| 10 | 0 | 0 | 0 | 0 | 77.3-140.7 |
| 11 | 0 | 0 | 0 | 0 | 55.3-109 |
| 12 | 0 | 0 | 0 | 0 | 69-105 |
| 13 | 0 | 0 | 0 | 0 | 68-126.3 |
| 14 | 0 | 0 | 0 | 0 | 69.7-167.7 |
| 15 | 0 | 0 | 0 | 0 | 87.3-141 |
| 16 | 0 | 0 | 0 | 0 | 49.3-115.7 |
| 17 | 0 | 0 | 0 | 0 | 73-149 |
| 18 | 0 | 0 | 0 | 0 | 74.3-139.3 |
| 19 | 0 | 0 | 0 | 0 | 68-129.7 |
| 20 | 0 | 0 | 0 | 0 | 60-118.3 |
| 21 | 0 | 0 | 0 | 0 | 62-92.7 |
| 22 | 1 | 1 | unk | unk | 69.3-143 |
| 23 | 0 | 0 | 0 | 0 | 66.7-86 |
| 24 | 1 | 1 | 13 | 13 | 71-195 |
| 25 | 0 | 0 | 0 | 0 | 60.3-133 |
| 26 | 0 | 0 | 0 | 0 | 89-123.7 |
| 27 | 0 | 0 | 0 | 0 | 50.7-133 |
| 28 | 0 | 0 | 0 | 0 | 66.7-108.7 |
| 29 | 0 | 0 | 0 | 0 | 57.3-116 |
| 30 | 0 | 0 | 0 | 0 | 60.3-129.3 |
| 31 | 1 | 1 | 15 | 15 | 76.7-104.3 |
| 32 | 0 | 0 | 0 | 0 | 85.3-126.3 |
| 33 | 0 | 0 | 0 | 0 | 60.3-104 |
| 34 | 1 | 1 | 9 | 9 | 68.3-123.7 |
| 35 | 0 | 0 | 0 | 0 | 74-127.3 |
| 36 | 0 | 0 | 0 | 0 | 69.7-116.7 |
| 37 | 1 | 1 | 11 | 11 | 71.3-119.3 |
| 38 | 0 | 0 | 0 | 0 | 81.7-131.7 |
| 39 | 0 | 0 | 0 | 0 | 57.7-124.7 |
| 40 | 0 | 0 | 0 | 0 | 60-104 |
| 41 | 1 | 1 | 8 | 8 | 44.7-93.7 |
| 42 | 0 | 0 | 0 | 0 | 61-129.7 |
| 43 | 0 | 0 | 0 | 0 | 66.7-137.7 |
| 44 | 0 | 0 | 0 | 0 | 89.3-139.3 |
| 45 | 0 | 0 | 0 | 0 | 69.3-130 |
| 46 | 1 | 1 | 15 | 15 | 59.7-88.3 |
| 47 | 0 | 0 | 0 | 0 | 70-128.3 |
| 48 | 0 | 0 | 0 | 0 | 74.7-134.3 |
| 49 | 0 | 0 | 0 | 0 | 77.3-107.7 |
| 50 | 1 | 1 | 38 | 34 | 70-109.7 |
| 51 | 0 | 0 | 0 | 0 | 67-112.3 |
| 52 | 0 | 0 | 0 | 0 | 66-104 |
| 53 | 0 | 0 | 0 | 0 | 66-130 |

TABLE 13

| | MAP @ | Na | Na | Cr | Cr | TBili |
|---|---|---|---|---|---|---|
| Donor | procurement | Peak | Proc | Peak | Proc | Peak |
| 1 | 78.7 | 167 | 146 | 0.7 | 0.5 | 1.3 |
| 2 | 113.3 | 173 | 146 | 3.9 | 3.7 | 1.7 |
| 3 | 106.3 | 157 | 151 | 1.08 | 0.74 | 1.2 |
| 4 | 84 | 162 | 155 | 1.1 | 0.9 | 1.0 |
| 5 | 71 | 165 | 131 | 1.4 | 2.9 | 1.7 |
| 6 | 104 | 151 | 149 | 0.9 | 0.9 | 1.5 |
| 7 | 80.7 | 140 | 134 | 8.3 | 6.4 | 1.1 |
| 8 | 93 | 160 | 149 | 2.7 | 2.5 | 4.9 |
| 9 | 115.7 | 191 | 154 | 2.7 | 2.7 | 1.6 |
| 10 | 114.3 | 169 | 150 | 1.6 | 1.4 | 1.7 |
| 11 | 55.3 | 154 | 151 | 0.8 | 0.7 | 0.7 |
| 12 | 86.3 | 152 | 144 | 6.7 | 3.9 | 0.9 |
| 13 | 102.3 | 155 | 149 | 1.79 | 1.0 | 1.8 |
| 14 | 91 | 160 | 130 | 1.1 | 0.5 | 2.9 |
| 15 | 94 | 188 | 147 | 2.71 | 1.61 | 1.3 |
| 16 | 111.7 | 152 | 151 | 1.1 | 0.9 | 2.2 |
| 17 | 106.7 | 152 | 150 | 1.3 | 0.6 | 1.1 |
| 18 | 105.7 | 148 | 148 | 3.5 | 2.7 | 3.2 |
| 19 | 122 | 158 | 145 | 1.1 | 0.8 | 0.4 |
| 20 | 101.7 | 160 | 157 | 2.17 | 1.09 | 2.6 |
| 21 | 78.7 | 147 | 154 | 6.67 | 5.59 | 0.7 |
| 22 | 96 | 175 | 151 | 2.4 | 1.5 | 1.8 |
| 23 | 71.7 | 137 | 136 | 0.85 | 0.52 | 1.1 |
| 24 | 88 | 159 | 150 | 1.1 | 1.1 | 0.7 |
| 25 | 65.3 | 179 | 164 | 2.0 | 2.0 | 2.4 |
| 26 | 81.7 | 151 | 136 | 2.6 | 0.6 | 0.7 |
| 27 | 97.3 | 159 | 149 | 2.2 | 2.0 | 0.6 |
| 28 | 75 | 148 | 148 | 2.4 | 1.3 | 0.8 |
| 29 | 95.7 | 169 | 145 | 1.4 | 0.8 | 0.9 |
| 30 | 71 | 164 | 157 | 1.49 | 1.07 | 1.8 |
| 31 | 91.7 | 167 | 157 | 1.3 | 1.1 | 0.5 |
| 32 | 105.7 | 156 | 153 | 2.7 | 2.7 | 3.6 |
| 33 | 83.7 | 167 | 136 | 1.0 | 1.0 | 1.2 |
| 34 | 100.7 | 150 | 146 | 2.4 | 2.1 | 1.0 |
| 35 | 70.7 | 166 | 140 | 1.1 | 1.0 | 0.7 |
| 36 | 112 | 161 | 144 | 0.8 | 0.5 | 1.8 |
| 37 | 101 | 150 | 141 | 5.2 | 5.2 | 1.5 |
| 38 | 93 | 149 | 139 | 0.7 | 0.76 | 2.1 |
| 39 | 91.7 | 171 | 146 | 0.99 | 0.99 | 0.8 |
| 40 | 85 | 173 | 138 | 2.48 | 2.16 | 0.6 |
| 41 | 85.3 | 156 | 136 | 4.2 | 3.6 | 0.9 |
| 42 | 84 | 174 | 154 | 1.2 | 0.7 | 4.4 |
| 43 | 90 | 169 | 138 | 1.4 | 0.9 | 0.8 |
| 44 | 90 | 151 | 145 | 1.4 | 1.4 | 0.9 |
| 45 | 116.3 | 165 | 141 | 2.8 | 2.0 | 1.3 |
| 46 | 72 | 153 | 153 | 1.38 | 1.38 | 0.5 |
| 47 | 102.7 | 168 | 139 | 0.7 | 0.6 | 0.6 |
| 48 | 84 | 159 | 145 | 1.7 | 1.0 | 1.5 |
| 49 | 111.7 | 160 | 153 | 1.6 | 1.4 | 0.8 |
| 50 | 99 | 149 | 142 | 4.4 | 4.1 | 1.3 |
| 51 | 76.3 | 146 | 144 | 9.0 | 8.4 | 2.4 |
| 52 | 81.7 | 172 | 157 | 3.9 | 3.9 | 2.4 |
| 53 | 130.7 | 156 | 151 | 2.14 | 1.94 | 3.0 |

TABLE 14

| | TBili | AST | AST | ALT | ALT |
|---|---|---|---|---|---|
| Donor | Proc | Peak | Proc | Peak | Proc |
| 1 | 1.0 | 77 | 32 | 59 | 42 |
| 2 | 1.1 | 45 | 28 | 21 | 21 |
| 3 | 1.0 | 1185 | 268 | 1293 | 106 |
| 4 | 0.9 | 109 | 49 | 194 | 87 |
| 5 | 1.7 | 54 | 482 | 29 | 103 |
| 6 | 0.6 | 55 | 20 | 61 | 33 |
| 7 | 0.6 | 31 | 31 | 15 | 11 |
| 8 | 3.7 | 111 | 54 | 49 | 40 |
| 9 | 1.6 | 74 | 74 | 126 | 50 |
| 10 | 1.7 | 216 | 41 | 209 | 107 |
| 11 | 0.3 | 28 | 21 | 40 | 40 |
| 12 | 0.4 | 370 | 130 | 197 | 131 |
| 13 | 1.3 | 273 | 170 | 78 | 76 |
| 14 | 1.6 | 128 | 34 | 100 | 53 |
| 15 | 0.9 | 43 | 35 | 38 | 40 |
| 16 | 1.2 | 59 | 29 | 14 | 15 |
| 17 | 0.6 | 44 | 18 | 33 | 59 |
| 18 | 1.5 | 77 | 24 | 32 | 14 |
| 19 | 1.1 | 244 | 19 | 165 | 36 |
| 20 | 0.9 | 60 | 94 | 150 | 26 |
| 21 | 0.4 | 31 | 23 | 24 | 15 |
| 22 | 1.1 | 619 | 22 | 973 | 169 |
| 23 | 1.6 | 137 | 42 | 72 | 59 |
| 24 | 0.4 | 41 | 39 | 37 | 37 |
| 25 | 0.8 | 49 | 49 | 38 | 38 |
| 26 | 0.9 | 38 | 24 | 34 | 23 |
| 27 | 0.5 | 40 | 44 | 24 | 24 |
| 28 | 0.8 | 720 | 727 | 200 | 227 |
| 29 | 0.5 | 70 | 23 | 30 | 30 |
| 30 | 1.1 | 127 | 25 | 235 | 80 |
| 31 | 0.4 | 87 | 26 | 74 | 21 |
| 32 | 3.2 | 37 | 28 | 27 | 11 |
| 33 | 1.1 | 36 | 35 | 37 | 37 |
| 34 | 0.2 | 163 | 163 | 158 | 158 |
| 35 | 0.6 | 33 | 12 | 15 | 11 |
| 36 | 0.9 | 82 | 37 | 49 | 27 |
| 37 | 0.7 | 1677 | 69 | 531 | 26 |
| 38 | 0.9 | 35 | 24 | 30 | 21 |
| 39 | 0.8 | 59 | 48 | 55 | 50 |
| 40 | 0.4 | 82 | 27 | 47 | 38 |
| 41 | 0.9 | 33 | 22 | 51 | 27 |
| 42 | 4.0 | 52 | 17 | 22 | 13 |
| 43 | 0.6 | 52 | 25 | 33 | 30 |
| 44 | 0.6 | 48 | 22 | 28 | 16 |
| 45 | 0.5 | 24 | 17 | 28 | 16 |
| 46 | 0.4 | 243 | 13 | 113 | 32 |
| 47 | 0.6 | 59 | 31 | 40 | 20 |
| 48 | 1.4 | 491 | 267 | 138 | 81 |
| 49 | 0.4 | 51 | 18 | 32 | 28 |
| 50 | 1.1 | 468 | 63 | 488 | 81 |
| 51 | 1.4 | 194 | 29 | 168 | 81 |
| 52 | 2.4 | 88 | 88 | 73 | 49 |
| 53 | 3.0 | 333 | 183 | 78 | 74 |

TABLE 15

| | INR | INR | pH @ | pH @ | pCO₂ | pCO₂ |
|---|---|---|---|---|---|---|
| Donor | Peak | Proc | Adm | Proc | @ Adm | @ Proc |
| 1 | 12.7 | 1.14 | 7.47 | 7.47 | 29 | 33 |
| 2 | 1.96 | 1.45 | 7.45 | 7.49 | 34.8 | 33.8 |
| 3 | 1.62 | 1.62 | 7.25 | 7.45 | 31 | 38 |
| 4 | 1.1 | 1.0 | 7.38 | 7.39 | 34.8 | 35.1 |
| 5 | 2.0 | 1.4 | 7.19 | 7.27 | 53 | 37.2 |
| 6 | 1.2 | 1.2 | 7.42 | 7.35 | 30 | 41 |
| 7 | 1.6 | 1.6 | 7.48 | 7.39 | 40 | 29 |
| 8 | 2.1 | 1.5 | 7.55 | 7.42 | 25 | 34 |
| 9 | 11.7 | 1.0 | 7.58 | 7.34 | 29.1 | 29.4 |
| 10 | 1.8 | 1.5 | 7.35 | 7.38 | 48 | 44.9 |
| 11 | 1.22 | 1.07 | 7.46 | 7.39 | 32.4 | 30 |
| 12 | 1.7 | 1.3 | 7.24 | 7.44 | 47.3 | 41.1 |
| 13 | 1.42 | 1.1 | 7.41 | 7.47 | 37 | 38 |
| 14 | 1.1 | 1.1 | 7.37 | 7.43 | 36 | 37 |
| 15 | 1.5 | 1.3 | 7.44 | 7.4 | 39 | 32 |
| 16 | 15 | 1.3 | 7.31 | 7.51 | 49 | 35 |
| 17 | 1.17 | 1.09 | 7.4 | 7.46 | 37.7 | 126 |
| 18 | 1.6 | 1.1 | 7.35 | 7.44 | 40.2 | 39.5 |
| 19 | 1.26 | 1.26 | 7.43 | 7.45 | 19.3 | 40.3 |
| 20 | 8.73 | 1.45 | 7.34 | 7.46 | 33 | 34 |
| 21 | 1.79 | 1.79 | 7.41 | 7.26 | 44 | 39 |
| 22 | 1.65 | 1.51 | 7.15 | 7.37 | 96.7 | 41.5 |
| 23 | 1.01 | 1.2 | 7.2 | 7.35 | 48.2 | 34 |
| 24 | 1.03 | 0.99 | 7.07 | 7.35 | 31 | 41 |
| 25 | 1.9 | 1.8 | 7.35 | 7.4 | 39 | 31 |
| 26 | 1.37 | 1.1 | 7.4 | 7.47 | 36.2 | 37.4 |
| 27 | 1.1 | 1.0 | 7.52 | 7.46 | 30.7 | 32 |
| 28 | 1.3 | 1.2 | 7.34 | 7.43 | 29.1 | 33.9 |
| 29 | 1.7 | 1.5 | 7.29 | 7.42 | 36.7 | 30 |
| 30 | 1.9 | 1.2 | 7.09 | 7.4 | 77 | 38 |
| 31 | 1.1 | 1.1 | 6.99 | 7.38 | 95.7 | 48 |
| 32 | 1.7 | 1.4 | 7.53 | 7.42 | 30.4 | 25.3 |
| 33 | 1.5 | 1.1 | 7.44 | 7.39 | 26 | 30 |
| 34 | 1.0 | 1.2 | 7.44 | 7.45 | 32 | 35 |
| 35 | 1.4 | 1.1 | 7.38 | 7.34 | 43 | 32 |
| 36 | 1.2 | 1.1 | 7.44 | 7.48 | 56 | 36 |
| 37 | 1.3 | 1.3 | 6.95 | 7.34 | 57 | 44 |
| 38 | 1.04 | 0.99 | 7.38 | 7.46 | 34 | 39 |
| 39 | 1.67 | 1.5 | 7.51 | 7.42 | 23.9 | 35.6 |
| 40 | 1.58 | 1.47 | 7.34 | 7.35 | 36.9 | 39 |
| 41 | 1.4 | 1.4 | 7.46 | 7.39 | 27.6 | 35.3 |
| 42 | 1.42 | 1.16 | 7.30 | 7.34 | 35.2 | 66 |
| 43 | 1.5 | 1.8 | 7.4 | 7.6 | 31.9 | 24 |
| 44 | 1.6 | 1.4 | 7.39 | 7.35 | 29 | 33 |
| 45 | 1.2 | 1.2 | 7.23 | 7.36 | 61 | 49 |
| 46 | 1.55 | 1.55 | 7.25 | 7.44 | 33 | 28 |
| 47 | 1.3 | 1.3 | 7.34 | 7.54 | 36.8 | 26.6 |
| 48 | 1.27 | 1.18 | 7.24 | 7.43 | 42 | 40.2 |
| 49 | 1.2 | 1.1 | 7.41 | 7.37 | 39 | 50 |
| 50 | 1.16 | 1.1 | 7.21 | 7.9 | 32.3 | 32.3 |
| 51 | 1.77 | 1.07 | 7.26 | 7.36 | 45 | 42.5 |
| 52 | 1.4 | 1.4 | 7.28 | 7.47 | 31 | 26 |
| 53 | 1.35 | 1.1 | 7.18 | 7.34 | 58 | 53 |

TABLE 16

| | | | | | | Liver |
|---|---|---|---|---|---|---|
| | | | | | | Abnormal |
| | pO2 | pO2 | HCO3 | HCO3 | | on |
| Donor | @ Adm | @ Proc | @ Adm | @ Proc | pO2 < 60 | Imaging |
| 1 | 151 | 525 | 22.8 | 25 | 0 | 0 |
| 2 | 228 | 194 | 23.5 | 25.3 | 0 | na |
| 3 | 603 | 542 | 13 | 26 | 0 | 1 |
| 4 | 195 | 175 | 20.8 | 21.2 | 0 | 0 |
| 5 | 362 | 90.5 | 19.4 | 16.5 | 0 | 1 |
| 6 | 140 | 135 | 19 | 22 | 0 | 0 |
| 7 | 269 | 99 | 30 | 17.6 | 0 | na |
| 8 | 68 | 141 | 21.9 | 22.1 | 0 | 1 |
| 9 | 49 | 92.5 | 26.7 | 21 | 1 | 0 |
| 10 | 178 | 334 | 27 | 26.8 | 0 | 0 |
| 11 | 478 | 107 | 32.4 | 18 | 0 | 0 |
| 12 | 199 | 64 | 19.5 | 28 | 0 | na |
| 13 | 101 | 240 | 23.5 | 27.7 | 0 | 0 |
| 14 | 448 | 121 | 20.1 | 24 | 0 | 1 |
| 15 | 389 | 588 | 26.5 | 19.8 | 0 | 0 |
| 16 | 485 | 430 | 21.6 | 28.1 | 0 | 0 |
| 17 | 163 | 126 | 22.8 | 31.4 | 0 | na |
| 18 | 421 | 496 | 21.5 | 26.3 | 0 | na |
| 19 | 297 | 345 | 12.8 | 27.7 | 0 | 0 |
| 20 | 321 | 174 | 18 | 25 | 0 | 0 |
| 21 | 120 | 204 | 28 | 17.5 | 0 | 0 |
| 22 | 83.7 | 98.9 | 32 | 24.2 | 0 | na |
| 23 | 124 | 95.2 | 18.7 | 18.5 | 0 | na |
| 24 | 168 | 120 | 22 | 22.6 | 0 | 0 |
| 25 | 370 | 461 | 21.5 | 19.2 | 0 | 0 |
| 26 | 116 | 161 | 22.2 | 26.6 | 0 | 0 |
| 27 | 97.9 | 501 | 24.4 | 22 | 0 | 0 |
| 28 | 195 | 147 | 15.4 | 22.2 | 0 | 0 |
| 29 | 167 | 398 | 17.8 | 21.4 | 0 | 0 |
| 30 | 47 | 119 | 23 | 23.5 | 1 | 1 |
| 31 | 437 | 98.3 | 23.4 | 28 | 0 | 1 |
| 32 | 110 | 156 | 25.5 | 16 | 0 | na |
| 33 | 443 | 124 | 17 | 17.8 | 0 | na |
| 34 | 172 | 89 | 21.3 | 24.2 | 0 | na |
| 35 | 107 | 147 | 24.9 | 16.9 | 0 | 0 |
| 36 | 84.9 | 136 | 38.8 | 26.8 | 0 | na |
| 37 | 205 | 158 | 12.5 | 24.3 | 0 | 0 |
| 38 | 418 | 183 | 20.1 | 27.7 | 0 | na |
| 39 | 309 | 170 | 18.3 | 22.4 | 0 | na |
| 40 | 78 | 151 | 19.9 | 21.2 | 0 | na |
| 41 | 371 | 143 | 19.4 | 20.7 | 0 | 0 |
| 42 | 48.7 | 95 | 17.2 | 35 | 1 | 0 |
| 43 | 249 | 553 | 19 | 23 | 0 | 0 |
| 44 | 183 | 110 | 17.7 | 18.2 | 0 | na |
| 45 | 174 | 130 | 25.5 | 27.7 | 0 | 0 |
| 46 | 540 | 166 | 15.9 | 21.9 | 0 | 0 |
| 47 | 135 | 132 | 19.3 | 23.6 | 0 | na |
| 48 | 78.8 | 120 | 17.6 | 26.3 | 0 | 0 |
| 49 | 105 | 141 | 24.9 | 29.1 | 0 | na |
| 50 | 309 | 121 | 12.7 | 24.9 | 0 | na |
| 51 | 35 | 191 | 20.4 | 23.5 | 1 | 0 |
| 52 | 548 | 119 | 14.1 | 18.1 | 0 | 0 |
| 53 | 355 | 118 | 20 | 28 | 0 | na |

What is claimed is:

1. A method for determining whether liver tissue will be acceptable for transplantation, the method comprising:
(a) measuring indocyanine green plasma disappearance rates (ICG-PDR) in a brain-dead donor prior to organ procurement; and
(b) designating liver tissue in the donor as acceptable for transplantation if the ICG-PDR is greater than about 18%.

2. The method of claim 1, further comprising designating the liver tissue as acceptable for transplantation if the ICG-PCR is greater than about 24%.

3. The method of claim 1, wherein ICG-PDR is measured non-invasively.

4. The method of claim 3, wherein ICG-PDR is measured using a PULSON LiMON liver function monitor.

5. A method of determining whether liver tissue will be acceptable for transplantation, the method comprising:
(a) measuring indocyanine green plasma disappearance rates (ICG-PDR) in a brain-dead donor prior to organ procurement;
(b) comparing measured ICG-PDR to a threshold; and
(c) designating liver tissue in the donor as acceptable for transplantation if the ICG-PDR exceeds the threshold.

6. The method of claim 5, wherein the threshold is about 18%.

7. The method of claim 5, wherein the threshold is about 24%.

8. The method of claim 5, wherein ICG-PDR is measured non-invasively.

9. The method of claim 8, wherein ICG-PDR is measured using a PULSON LiMON liver function monitor.
